(19) United States
(12) Plant Patent Application Publication
(10) Pub. No.: US 2013/0318665 P1
(43) Pub. Date: Nov. 28, 2013

(54) Raspberry plant - Dolomia Plus Cultivar

(50) ;

(71) Applicant: SANT'ORSOLA SOCIETA' COOPERATIVA AGRICOLA (Italian Farmers Cooperative), (US)

(72) Inventors: Marco Giacomelli, Vigolo Vattaro (IT); Alessandra Viliotti, Vigolo Vattaro (IT)

(21) Appl. No.: 13/815,684

(22) Filed: Mar. 14, 2013

(30) Foreign Application Priority Data
May 25, 2012 (IT) CPVO 20121083

(51) Int. Cl. A01H 5/00

(52) U.S. Cl. PLT/204

(57) ABSTRACT

The raspberry cultivar “Dolomia Plus” was obtained by crossing of the varieties Tulameen and Polka at Vigolo Vattaro Trento, Italy. The new cultivar is a primocane variety producing two crops per year, wherein the fruits ripen on current season's cane in autumn. The resulting fruits are conical and medium red in coloration.

### GENUS AND SPECIES

Rubus idaeus L.

### VARIETY DENOMINATION

“Dolomia Plus”

### BACKGROUND OF THE NEW VARIETY

A new and distinct cultivar of red raspberry (Rubus idaeus L.) hereinafter referred to by the cultivar denomination “Dolomia Plus” is disclosed. The new variety “Dolomia Plus” was obtained from the cross by seedling of individual raspberry plants of the varieties Tulameen and Polka.

The crossing for the new cultivar occurred on year 2004 in Vigolo Vattaro (Trento-Italy) and it has been subject to selection on year 2005 and then tested since year 2007. The characteristics of the new cultivar have been found stable and have been transmitted without change through succeeding (more than 6 years) asexual propagations.

### SUMMARY OF THE INVENTION

The most outstanding and distinguishing characteristics of the “Dolomia Plus” cultivar are hereunder reported.
Primocane raspberry plant;
Good fruit colour and flavour;
Medium and firm red berries.

The new cultivar can be propagated by: the use of suckers produced in a spawn-bed, root cutting and use of tissue culture techniques. The root cutting is to be preferred.

### BRIEF DESCRIPTION OF THE PHOTOGRAPHS

The new red raspberry cultivar “Dolomia Plus” is illustrated by the accompanying photographs showing typical specimens of the new variety in colour as true as reasonably possible to make the same colour illustrations of this character by conventional photographic procedures.

FIG. 1 is a view showing a row of raspberry plants.

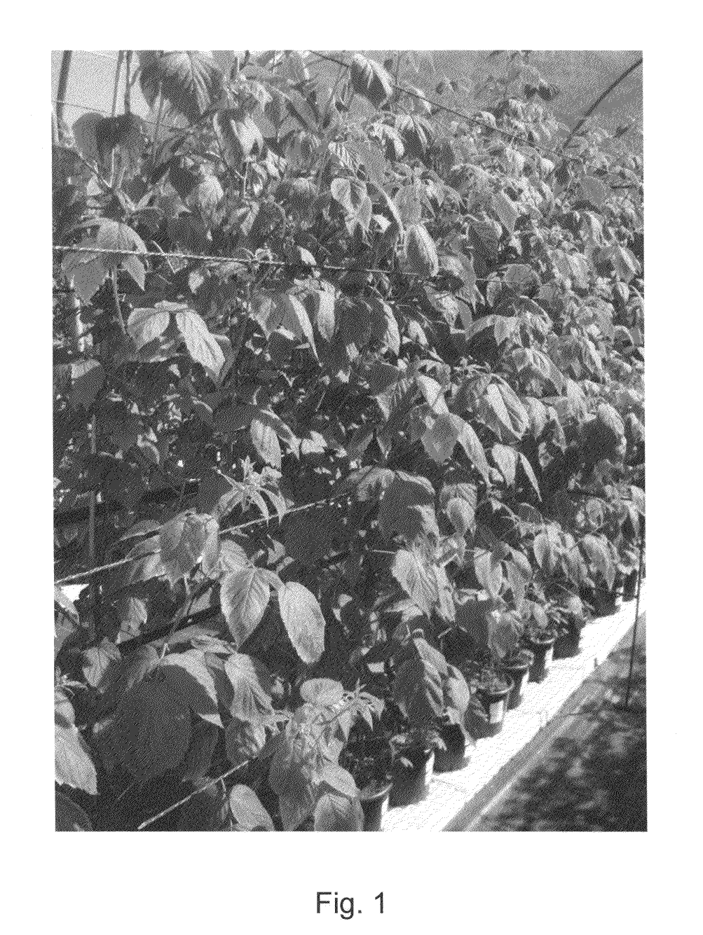

FIG. 2 is a view showing the fruit size, shape and colour at ripening.

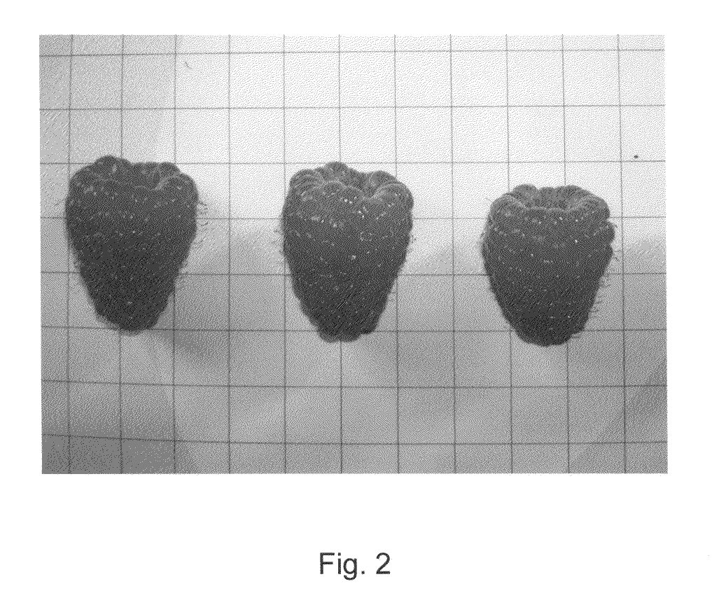

FIG. 3 is a view showing both the ripe and immature fruit size and shape.

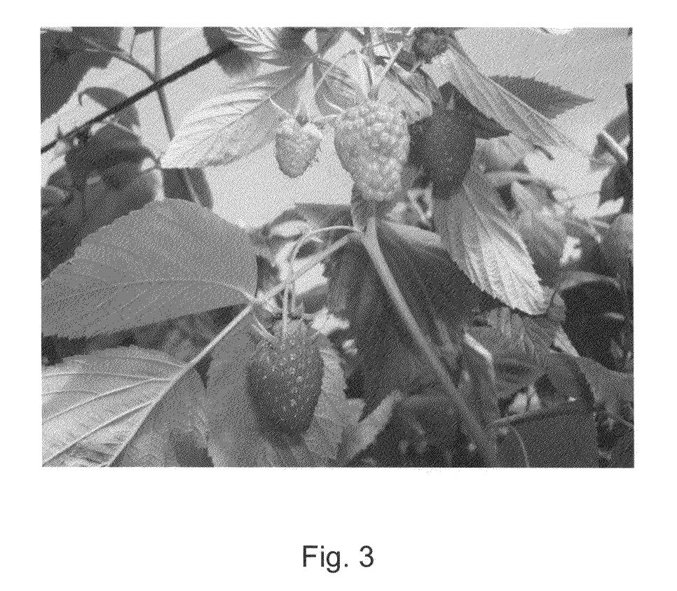

### DETAILED DESCRIPTION

The following detailed description set forth the distinctive characteristics of the new variety “Dolomia Plus”. The performance of new cultivar for retention of its distinctive characteristics has been evaluated through successive asexual propagations by root cutting in the Applicant's setting in Trentino (Italy) under controlled conditions of temperature (minimal temperature 15° C. and maximal temperature 28° C.) in greenhouse. The growing was the out of soil using peat as substrate.

The plant assessment was performed for 7 years.
Classification:
   Family.—Rosaceae;
   Botanical.—Rubus idaeus L.
   Common name.—Raspberry.
Parentage: Tulameen×Polka.
Plant:
   General habit.—upright canes; strong vigor; very good roots system; adapted to medium winter chill conditions such as those of Northern Italy; high productivity in autumn.
   Number of current season's shoots.—few to medium.
   Very young shoot.—anthocyanin coloration of apex during rapid growth present.
   Dormant cane colour.—brown.
   Spines.—present.
   Leaves.—bright green on the superior surface and light green on the inferior surface; concave; odd-pinnate and subdivided in 3-5 ovate and serrate small leafs with medium rugosity.
   Bloom.—on the current season's cane is early to medium and the flowers are typical of raspberry and the blooming is white and begins in medium season.
Fruit:
   Predominant shape.—conical.
   Weight.—5.0-6.0 g (approximately).
   Size.—ratio length/width 2.8 cm/2.3 cm (approximately).
   Colour of ripe fruit.—medium red.
   Firmness.—high.
   Main bearing time.—only on current year's cane in autumn; (productivity: 55% in autumn time, 45% in spring time).
   Time of beginning the fruit ripening on current season's cane.—early to medium; in spring time the fruit ripening time is similar to Tulameen.
   Storage capacity.—good shelf-life, good quality fruits adapted to fresh and frozen market.
Pest and disease resistance: Strong canes resistant to pests and diseases; the plants needs few spry treatments for the diseases and pests control. The new cultivar “Dolomia Plus” is similar to the commercial variety “Polka” as shown in Table 1.

TABLE 1
Comparison between “Dolomia Plus” and “Polka”

| Characteristic | “Dolomia Plus” | “Polka” |
|---|---|---|
| fruit color | bright red | dark red |
| fruit size | big | medium |
| fruit flavor | high | medium |
| plant vigor | strong | medium |

What is claimed:

1. A new and distinct cultivar of raspberry plant as substantially herein shown and described.
